(19) United States
(12) Patent Application Publication
(10) Pub. No.: US 2019/0156483 A1
(43) Pub. Date: May 23, 2019

(54) IMAGE PROCESSING APPARATUS AND IMAGE PROCESSING METHOD

(71) Applicant: OLYMPUS CORPORATION, Tokyo (JP)

(72) Inventors: Takashi KONO, Tokyo (JP); Yamato KANDA, Tokyo (JP); Takehito HAYAMI, Tokyo (JP)

(73) Assignee: OLYMPUS CORPORATION, Tokyo (JP)

(21) Appl. No.: 16/256,425

(22) Filed: Jan. 24, 2019

Related U.S. Application Data
(63) Continuation of application No. PCT/JP2016/071770, filed on Jul. 25, 2016.

(51) Int. Cl. G06T 7/00; A61B 1/00; A61B 1/04; G06T 7/11

(52) U.S. Cl. CPC G06T 7/0014; A61B 1/00009; G06T 2207/10068; G06T 7/11; A61B 1/04

(57) ABSTRACT

An image processing apparatus including a processor comprising hardware, wherein the processor is configured to execute: analyzing a characteristic of a pathologic region included in individual endoscopic images of an endoscopic image group arranged in chronological order; setting, based on the characteristic of the pathologic region, an extraction condition for extracting one or more endoscopic images appropriate for diagnosis from the endoscopic image group; and extracting, based on the extraction condition, one or more endoscopic images having image quality appropriate for diagnosis from the endoscopic image group. When performing the analysis of the characteristic of the pathologic region, the processor classifies the pathologic region into a preset class of malignant degree.

### CROSS-REFERENCE TO RELATED APPLICATION

This application is a continuation of International Application No. PCT/JP2016/071770, filed on Jul. 25, 2016, the entire contents of which are incorporated herein by reference.

### BACKGROUND

The present disclosure relates to an image processing apparatus and an image processing method.

Japanese Laid-open Patent Publication No. 2004-24559 discloses a technology of extracting a display image from an instructed image periphery of a user using image quality and operation information as indices. This may solve bother of repeatedly performing capturing so as to obtain a high-quality image, because a freeze manipulation in an ultrasonograph deteriorates image quality due to blurring, unsharpness, and the like that are attributed to a posture change of an ultrasound probe that are caused by holding of an the ultrasound probe by the hand of a diagnostician, aspiration, a body posture change, and the like. Specifically, after a plurality of chronological ultrasound images are stored, a freeze image is set according to an instruction of the user, a plurality of candidate images having a relationship of approaching temporally the freeze image are selected, and a display image is selected using reference information such as image quality and an operation that accompanies the plurality of candidate images, as feature data (index).

### SUMMARY

An image processing apparatus according to one aspect of the present disclosure includes a processor comprising hardware, wherein the processor is configured to execute: analyzing a characteristic of a pathologic region included in individual endoscopic images of an endoscopic image group arranged in chronological order; setting, based on the characteristic of the pathologic region, an extraction condition for extracting one or more endoscopic images appropriate for diagnosis from the endoscopic image group; and extracting, based on the extraction condition, one or more endoscopic images having image quality appropriate for diagnosis from the endoscopic image group, wherein, when performing the analysis of the characteristic of the pathologic region, the processor acquires pathologic region information representing coordinate information of a pathologic region in each endoscopic image of the endoscopic image group, the pathologic region information being generated by detecting a pathologic region by a pathologic region detection device from each endoscopic image of the endoscopic image group, acquires, based on the pathologic region information, pathologic region presence information representing whether a pathologic region having an area equal to or larger than a preset predetermined value is included in each endoscopic image, calculates, based on the pathologic region information, pathology characteristic information representing a characteristic of the pathologic region, and classifies the pathologic region into a preset class of malignant degree.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a configuration of an image processing apparatus according to a first embodiment;

FIG. 2 is a flowchart illustrating an overview of processing executed by the image processing apparatus according to the first embodiment;

FIG. 3 is a flowchart illustrating an overview of pathologic region characteristic analysis processing in FIG. 2;

FIG. 4 is a flowchart illustrating an overview of extraction condition setting processing in FIG. 2;

FIG. 5 is a block diagram illustrating a configuration of a pathology characteristic information calculation unit according to a first modified example of the first embodiment;

FIG. 6 is a block diagram illustrating a configuration of a gazing operation determination unit according to the first modified example of the first embodiment;

FIG. 7 is a block diagram illustrating a configuration of a base point image setting unit according to the first modified example of the first embodiment;

FIG. 8 is a block diagram illustrating a configuration of an edge point section setting unit according to the first modified example of the first embodiment;

FIG. 9 is a block diagram illustrating a configuration of a pathology characteristic information calculation unit according to a second modified example of the first embodiment;

FIG. 10 is a block diagram illustrating a configuration of a gazing operation determination unit according to the first modified example of the second embodiment;

FIG. 11 is a block diagram illustrating a configuration of a pathologic region analysis unit according to a third modified example of the first embodiment;

FIG. 12 is a flowchart illustrating an overview of pathologic region characteristic analysis processing executed by the pathologic region analysis unit according to the third modified example of the first embodiment;

FIG. 13 is a block diagram illustrating a configuration of a pathologic region analysis unit according to a fourth modified example of the first embodiment;

FIG. 14 is a flowchart illustrating an overview of pathologic region characteristic analysis processing executed by the pathologic region analysis unit according to the fourth modified example of the first embodiment;

FIG. 15 is a block diagram illustrating a configuration of an arithmetic unit according to a second embodiment;

FIG. 16 is a flowchart illustrating an overview of processing executed by the image processing apparatus according to the second embodiment;

FIG. 17 is a flowchart illustrating an overview of pathologic region characteristic analysis processing in FIG. 16;

FIG. 18 is a flowchart illustrating an overview of extraction condition setting processing in FIG. 16;

FIG. 19 is a flowchart illustrating an overview of endoscopic image extraction processing in FIG. 16;

FIG. 20 is a block diagram illustrating a configuration of an arithmetic unit according to a third embodiment;

FIG. 21 is a flowchart illustrating an overview of processing executed by the image processing apparatus according to the third embodiment;

FIG. 22 is a flowchart illustrating an overview of pathologic region characteristic analysis processing in FIG. 21;

FIG. 23 is a flowchart illustrating an overview of extraction condition setting processing in FIG. 21; and

FIG. 24 is a flowchart illustrating an overview of endoscopic image extraction processing in FIG. 21.

### DETAILED DESCRIPTION OF THE PREFERRED EMBODIMENTS

Hereinafter, an image processing apparatus, an image processing method, and a program according to embodiments of the present disclosure will be described with reference to the drawings. In addition, the present disclosure is not limited by these embodiments. In addition, in the description of the drawings, the same parts are assigned the same signs.

### First Embodiment

### Configuration of Image Processing Apparatus

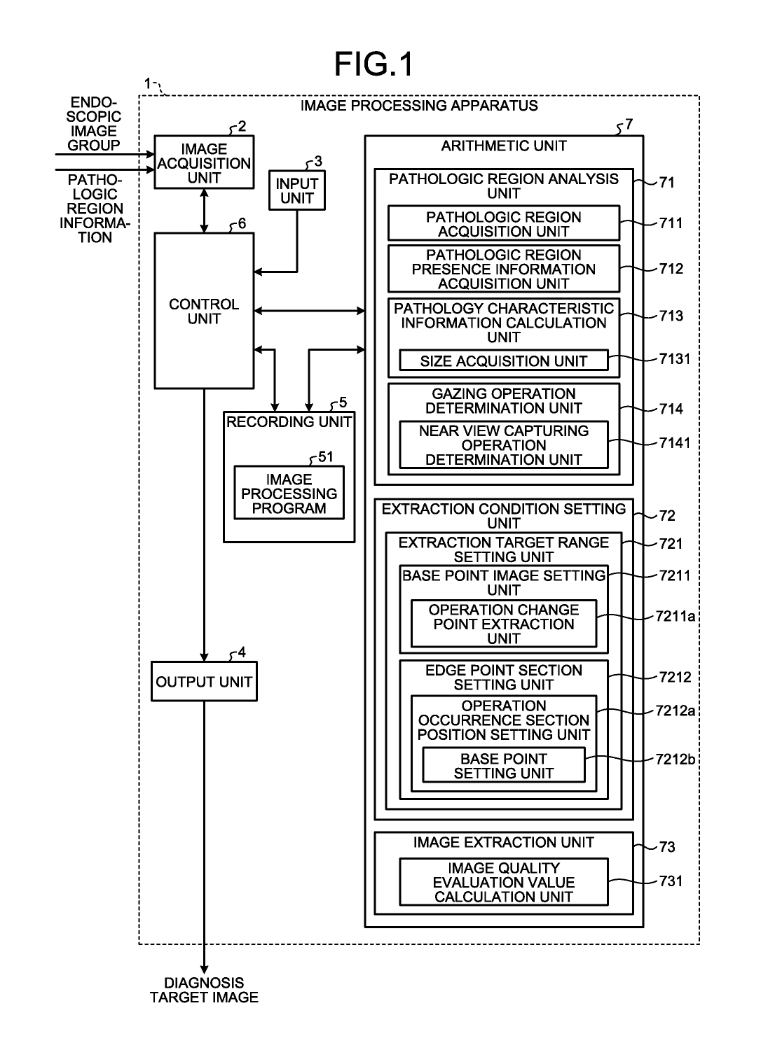

FIG. 1 is a block diagram illustrating a configuration of an image processing apparatus according to a first embodiment. As an example, an image processing apparatus 1 according to this first embodiment is an apparatus that extracts a high-quality endoscopic image optimum for diagnosis from an endoscopic image group (moving image data and time-series image group) consecutively captured by an endoscope (endoscopic scope such as a flexible endoscope and a rigid endoscope) or a capsule endoscope (hereinafter, these are collectively and simply referred to as an “endoscope”) and arranged in chronological order. In addition, normally, an endoscopic image is a color image having a pixel level (pixel value) corresponding to a wavelength component of red (R), green (G), or blue (B) at each pixel position. In addition, hereinafter, a pathologic region is a specific region including pathology or an abnormal portion such as bleeding, reddening, congealed blood, tumor, erosion, ulcer, aphtha, and chorionic abnormality, as a specific region, that is to say, an abnormal region.

The image processing apparatus 1 illustrated in FIG. 1 includes an image acquisition unit 2 that acquires pathologic region information representing coordinate information of a pathologic region detected by a pathologic region detection device (e.g. machine learning device such as Deep Learning) from an endoscopic image group captured by an endoscope, an input unit 3 that receives an input signal input by a manipulation from the outside, an output unit 4 that outputs a diagnosis target image optimum for diagnosis among the endoscopic image group, to the outside, a recording unit 5 that records the endoscopic image group acquired by the image acquisition unit 2 and various programs, a control unit 6 that controls operations of the entire image processing apparatus 1, and an arithmetic unit 7 that performs predetermined image processing on the endoscopic image group.

The image acquisition unit 2 is appropriately formed according to the mode of a system including an endoscope. For example, when a portable recording medium is used for transferring an endoscopic image group (moving image data, image data) and pathologic region information with an endoscope, the image acquisition unit 2 is formed as a reader device that has the recording medium detachably attached thereto and reads the recorded endoscopic image group and pathologic region information. In addition, when a server that records an endoscopic image group captured by an endoscope and pathologic region information is used, the image acquisition unit 2 is formed by a communication device that can bi-directionally communicate with the server, or the like, and acquires the endoscopic image group and the pathologic region information by performing data communication with the server. Furthermore, in addition, the image acquisition unit 2 may be formed by an interface device to which an endoscopic image group and pathologic region information are input from an endoscope via a cable, or the like.

The input unit 3 is implemented by an input device such as a keyboard, a mouse, a touch panel, and various switches, for example, and outputs an input signal received according to a manipulation from the outside, to the control unit 6.

Under the control of the control unit 6, the output unit 4 outputs a diagnosis target image extracted by the calculation of the arithmetic unit 7, to an external display device, or the like. In addition, the output unit 4 is formed using a display panel such as a liquid crystal or an organic Electro Luminescence (EL), and may display various images including a diagnosis target image by the calculation of the arithmetic unit 7.

The recording unit 5 is implemented by various IC memories such as a flash memory, a read only memory (ROM), and a random access memory (RAM), and a hard disc that is incorporate or connected by a data communication terminal, or the like. Aside from the endoscopic image group acquired by the image acquisition unit 2, the recording unit 5 records programs for operating the image processing apparatus 1 and causing the image processing apparatus 1 to execute various functions, data used in the execution of the programs, and the like. For example, the recording unit 5 records an image processing program 51 for extracting one or more endoscopic images optimum for diagnosis from an endoscopic image group, various types of information used in the execution of the program, and the like.

The control unit 6 is formed using a general-purpose processor such as a central processing unit (CPU), or a dedicated processor such as various arithmetic circuits that execute specific functions such as an application specific integrated circuit (ASIC) and a field programmable gate array (FPGA). When the control unit 6 is a general-purpose processor, the control unit 6 performs instructions, data transfer, and the like to units constituting the image processing apparatus 1, by reading various programs stored in the recording unit 5, and comprehensively controls operations of the entire image processing apparatus 1. In addition, when the control unit 6 is a dedicated processor, the processor may independently execute various types of processing, or the processor and the recording unit 5 may execute various types of processing in cooperation or in combination, by using various data stored in the recording unit 5, and the like.

The arithmetic unit 7 is formed using a general-purpose processor such as a CPU, or a dedicated processor such as various arithmetic circuits that executes specific functions such as an ASIC or an FPGA. When the arithmetic unit 7 is a general-purpose processor, the arithmetic unit 7 executes image processing of extracting an endoscopic image optimum for diagnosis from the acquired endoscopic image group arranged in chronological order, by reading the image processing program 51 from the recording unit 5. In addition, when the arithmetic unit 7 is a dedicated processor, the processor may independently execute various types of processing, or the processor and the recording unit 5 may execute image processing in cooperation or in combination, by using various data stored in the recording unit 5, and the like.

### Detailed Configuration of Arithmetic Unit

Next, a detailed configuration of the arithmetic unit 7 will be described.

The arithmetic unit 7 includes a pathologic region analysis unit 71, an extraction condition setting unit 72, and an image extraction unit 73.

The pathologic region analysis unit 71 receives input of an endoscopic image group acquired by the image acquisition unit 2 via the control unit 6 or the recording unit 5, and pathologic region information representing coordinate information of a pathologic region in each endoscopic image, and analyzes features and characteristics of a pathologic region included in individual endoscopic images. The pathologic region analysis unit 71 includes a pathologic region acquisition unit 711, a pathologic region presence information acquisition unit 712, a pathology characteristic information calculation unit 713, and a gazing operation determination unit 714.

The pathologic region acquisition unit 711 acquires an endoscopic image group acquired by the image acquisition unit 2 via the control unit 6 or the recording unit 5, and pathologic region information representing coordinate information of a pathologic region in each endoscopic image.

Based on pathologic region information of each endoscopic image, the pathologic region presence information acquisition unit 712 acquires pathologic region presence information as to whether a pathologic region having an area equal to or larger than a preset predetermined value is included.

Based on the pathologic region information, the pathology characteristic information calculation unit 713 calculates pathology characteristic information representing a characteristic of a pathologic region. In addition, the pathology characteristic information calculation unit 713 includes a size acquisition unit 7131 that acquires size information of a pathologic region based on pathologic region information when pathologic region presence information includes information representing that a pathologic region having an area equal to or larger than a preset predetermined value is included (hereinafter, referred to as a “case of present determination”).

The gazing operation determination unit 714 determines a gazing operation on a pathologic region based on the pathology characteristic information. In addition, the gazing operation determination unit 714 includes a near view capturing operation determination unit 7141 that determines that gazing and near view imaging are being performed, when pathologic region presence information represents present determination and size information in pathology characteristic information represents a preset predetermined value or more.

The extraction condition setting unit 72 sets an extraction condition based on the characteristic (feature) of a pathologic region. The extraction condition setting unit 72 includes an extraction target range setting unit 721.

Based on the characteristic (feature) of the pathologic region, the extraction target range setting unit 721 sets a range between a base point and edge points decided based on the base point, as an extraction target range. In addition, the extraction target range setting unit 721 includes a base point image setting unit 7211 that sets an endoscopic image at a specific operation position as a reference image based on operation information in the characteristic (feature) of the pathologic region, and an edge point section setting unit 7212 that sets endoscopic images at specific operation positions preceding and following the base point image as edge point images, and sets a section from the base point image to the edge point images, based on operation information in the characteristic (feature) of the pathologic region.

The base point image setting unit 7211 includes an operation change point extraction unit 7211a that sets, as a base point image, an endoscopic image near a position at which a specific operation switches to another operation after the specific operation has continued for a preset predetermined section.

The edge point section setting unit 7212 includes an operation occurrence section position setting unit 7212a that sets a section up to an image where a specific operation occurs. Furthermore, in addition, the operation occurrence section position setting unit 7212a includes a base point setting unit 7212b that sets, as edge point images, base point images preceding and following the base point image, in a section in which pathologic region presence information represents present determination.

Based on an extraction condition, the image extraction unit 73 extracts one or more endoscopic images, each having image quality appropriate for diagnosis (image quality satisfying a predetermined condition). The image extraction unit 73 includes an image quality evaluation value calculation unit 731 that calculates an evaluation value corresponding to the image quality of a pathologic region.

### Processing of Image Processing Apparatus

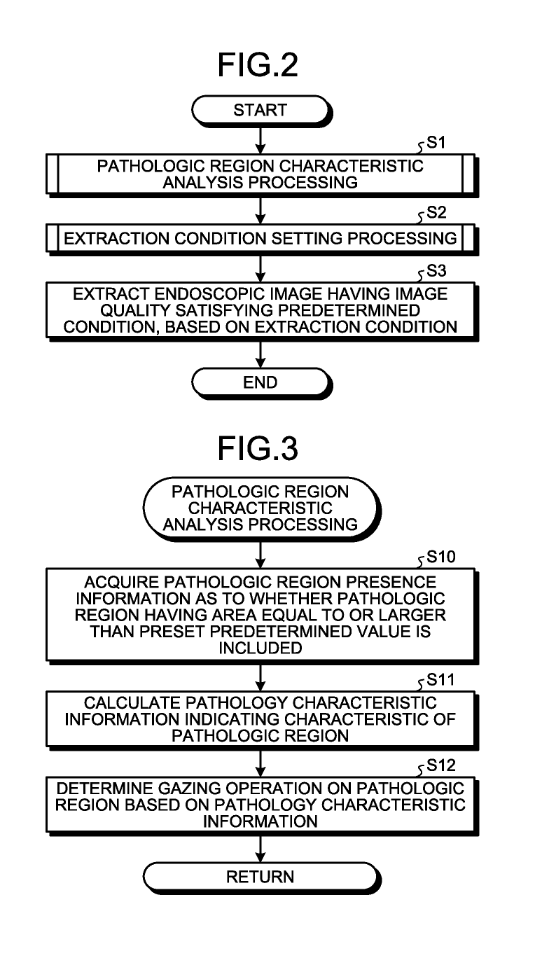

Next, an image processing method executed by the image processing apparatus 1 will be described. FIG. 2 is a flowchart illustrating an overview of processing executed by the image processing apparatus 1.

As illustrated in FIG. 2, the pathologic region analysis unit 71 acquires an endoscopic image group recorded in the recording unit 5 and pathologic region information, and executes pathologic region characteristic analysis processing of analyzing the characteristic (feature) of a pathologic region in each endoscopic image (Step S1). After Step S1, the image processing apparatus 1 advances the processing to Step S2 to be described later.

FIG. 3 is a flowchart illustrating an overview of the pathologic region characteristic analysis processing in Step S1 in FIG. 2. As illustrated in FIG. 3, based on an endoscopic image group being input information acquired from the recording unit 5 by the pathologic region acquisition unit 711, and pathologic region information having coordinate information of the pathologic region, the pathologic region presence information acquisition unit 712 acquires pathologic region presence information as to whether a pathologic region having an area equal to or larger than a preset predetermined value is included, and performs determination (Step S10). Specifically, the pathologic region presence information acquisition unit 712 determines whether pathologic region information includes coordinate information of a pathologic region and information (flag) indicating a pathologic region having an area equal to or larger than a preset predetermined value.

Subsequently, based on the pathologic region information, the pathology characteristic information calculation unit 713 calculates pathology characteristic information representing a characteristic of a pathologic region (Step S11). Specifically, when pathologic region presence information represents present determination, the size acquisition unit 7131 acquires size information of a pathologic region based on pathologic region information.

After that, the gazing operation determination unit 714 determines a gazing operation on a pathologic region based on the pathology characteristic information (Step S12). Specifically, the near view capturing operation determination unit 7141 determines that gazing is being performed, when pathologic region presence information represents present determination, and determines that near view imaging is being performed, when size information in pathology characteristic information is a preset predetermined value or more. After Step S12, the image processing apparatus 1 returns to a main routine in FIG. 2. Through the above processing, the pathologic region analysis unit 71 outputs operation information as the characteristic of a pathologic region to the extraction condition setting unit 72.

Referring back to FIG. 2, the description subsequent to Step S2 will be continued.

In Step S2, the extraction condition setting unit 72 executes, on an endoscopic image group, extraction condition setting processing of setting an extraction target range of extracting a base point and edge points decided based on the base point, based on the characteristic (feature) of the pathologic region.

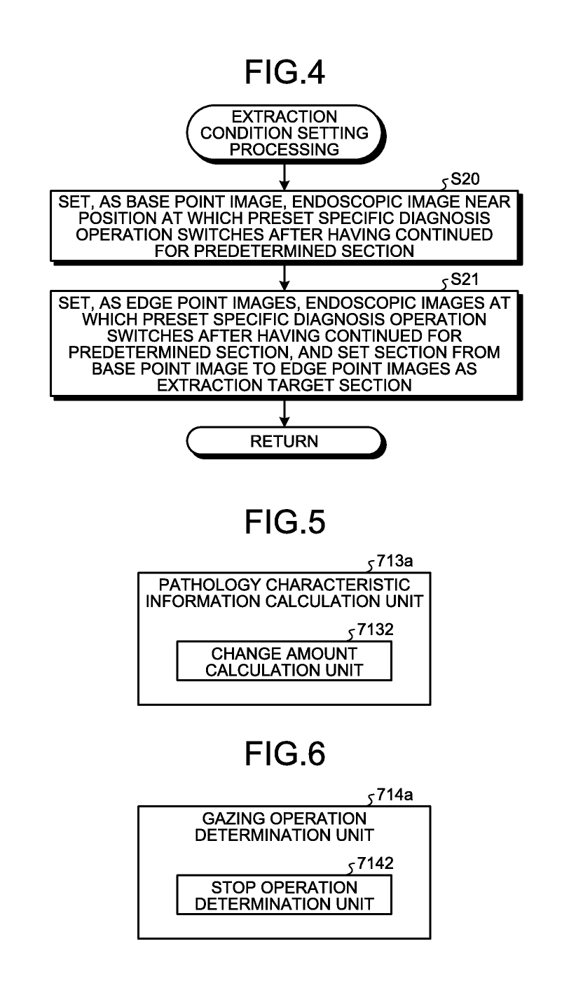

FIG. 4 is a flowchart illustrating an overview of the extraction condition setting processing in Step S2 in FIG. 2. As illustrated in FIG. 4, first, based on operation information in the characteristic (feature) of a pathologic region, the extraction target range setting unit 721 sets an endoscopic image at a specific operation position as a base point image (Step S20). Specifically, the base point image setting unit 7211 sets, as a base point image, an endoscopic image near a position at which a specific operation switches to another operation after the specific operation has continued for a preset predetermined section. More specifically, based on operation information, the operation change point extraction unit 7211a determines whether gazing is being performed and determines whether near view imaging is being performed, and sets, as a base point image, an endoscopic image that is located near a timing at which gazing switches to non-gazing, and near a timing at which near view imaging switches to distant view imaging. Here, near the timing refers to a time within a predetermined range from the timing at which gazing switches to non-gazing, and is one second, for example. In addition, near the position at which the operation switches to another operation refers to a time within a predetermined range from the position at which the operation switches to another operation, and is one second, for example.

Subsequently, based on operation information in the characteristic (feature) of a pathologic region, the edge point section setting unit 7212 sets endoscopic images at specific operation positions preceding and following the base point image as edge point images, and sets a section from the base point image to the edge point images (Step S21). Specifically, the operation occurrence section position setting unit 7212a sets a section up to an image where a specific operation occurs. More specifically, the base point setting unit 7212b sets, as an edge point image, an endoscopic image at a timing at which a diagnosis operation switches after a preset specific diagnosis operation has continued, and sets a section from the base point image to the edge point image. After Step S21, the image processing apparatus 1 returns to the aforementioned main routine in FIG. 2. Through the above processing, the extraction condition setting unit 72 outputs information of an extraction target range to the image extraction unit 73.

Referring back to FIG. 2, the description subsequent to Step S3 will be continued.

In Step S3, the image extraction unit 73 extracts an endoscopic image having predetermined condition image quality, based on the extraction condition. Specifically, the image quality evaluation value calculation unit 731 extracts an endoscopic image while assuming, as a pixel value, at least any one of a color shift amount, sharpness, and an effective region area in a surface structure. Here, regarding a color shift amount, the image quality evaluation value calculation unit 731 calculates a representative value (average value, etc.) of saturation information calculated from the entire image for the base point image, regards an endoscopic image having a smaller value as compared with the representative value of saturation information of the base point image, as an endoscopic image having a smaller color shift amount, and calculates an evaluation value regarding image quality, to be higher. In addition, regarding sharpness, the image quality evaluation value calculation unit 731 regards an endoscopic image having a larger value as compared with sharpness information of the base point image, as an endoscopic image having stronger sharpness, and calculates an evaluation value regarding image quality, to be higher. In addition, the image quality evaluation value calculation unit 731 calculates an evaluation value regarding image quality, to be higher as an effective region area becomes larger. Subsequently, the image extraction unit 73 extracts a high-quality image by extracting an image falling within a predetermined range on a feature data space of an image quality evaluation value, based on a calculated evaluation value.

Here, in JP 2004-24559 A described above, as feature data used when an image is selected from a reference range instructed by the user, image quality or an operation described in reference information is applied, and solution for reducing a burden on the user in instructing an extraction target range and the number of images to be extracted has not been mentioned. For example, in an intraluminal image of an endoscope, an operation change of a subject is larger and a pathologic region frequently goes into and out of a captured range, and in such a scene that a fluctuation of the subject is larger, the user is assumed to fail in instructing a freeze timing and setting an approach range of a freeze image, and a high-quality image has not been always extracted. In contrast to this, according to the first embodiment, analysis of the characteristic (feature) of a pathologic region that has been obtained as input information is performed, an extraction condition is set based on the characteristic (feature) of the pathologic region, and a high-quality image is extracted from a reference range image based on the extraction condition, whereby an endoscopic image appropriate for diagnosis can be extracted from an endoscopic image group arranged in chronological order.

### First Modified Example of First Embodiment

Next, a first modified example of the first embodiment will be described. The first modified example of this first embodiment is different in the configurations of the pathology characteristic information calculation unit 713, the gazing operation determination unit 714, the base point image setting unit 7211, and the edge point section setting unit 7212 according to the aforementioned first embodiment. Hereinafter, a pathology characteristic information calculation unit, a gazing operation determination unit, a base point image setting unit, and an edge point section setting unit according to the first modified example of this first embodiment will be described. In addition, the same configurations as those in the image processing apparatus 1 according to the aforementioned first embodiment are assigned the same sings, and the description thereof will be omitted.

FIG. 5 is a block diagram illustrating a configuration of a pathology characteristic information calculation unit according to the first modified example of the first embodiment. Based on the pathologic region information, a pathology characteristic information calculation unit 713a illustrated in FIG. 5 calculates pathology characteristic information representing a characteristic of a pathologic region. In addition, the pathology characteristic information calculation unit 713a includes a change amount calculation unit 7132 that calculates a change amount between pathologic regions with an endoscopic image adjacent to an endoscopic image of interest in chronological order, when pathologic region presence information represents present determination. Here, the change amount is an area size obtained by subtracting an area of logical product from an area of logical sum of two pieces of pathologic region information of an image of interest and an endoscopic image that the image of interest approaches.

FIG. 6 is a block diagram illustrating a configuration of a gazing operation determination unit according to the first modified example of the first embodiment. A gazing operation determination unit 714a illustrated in FIG. 6 determines a gazing operation on a pathologic region based on the pathology characteristic information. In addition, the gazing operation determination unit 714a includes a stop operation determination unit 7142 that stops when a change amount in pathology characteristic information is less than a preset predetermined value.

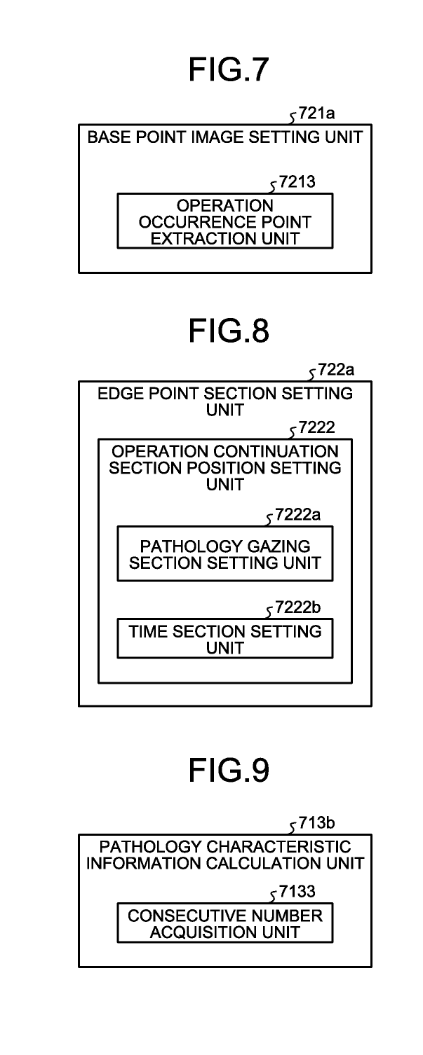

FIG. 7 is a block diagram illustrating a configuration of a base point image setting unit according to the first modified example of the first embodiment. Based on operation information in the characteristic (feature) of a pathologic region, a base point image setting unit 721a illustrated in FIG. 7 sets an endoscopic image at a specific operation position as a base point image. Specifically, when the operation information is information regarding moving or stop, the base point image setting unit 721a sets, as a base point image, an endoscopic image located before a timing at which stop switches to moving. In addition, the base point image setting unit 721a includes an operation occurrence point extraction unit 7213 that sets, as a base point image, a point at which a specific diagnosis operation occurs. Specifically, when operation information is information regarding a manipulation operation, the operation occurrence point extraction unit 7213 sets, as base point images, endoscopic images at start and end points of the manipulation of a manipulator at an image acquisition operation.

FIG. 8 is a block diagram illustrating a configuration of an edge point section setting unit according to the first modified example of the first embodiment. Based on operation information in image quality appropriate for characteristic (feature) diagnosis of a pathologic region, an edge point section setting unit 722a illustrated in FIG. 8 sets endoscopic images at specific operation positions preceding and following the base point image as edge point images, and sets a section from the base point image to the edge point images. In addition, the edge point section setting unit 722a includes an operation continuation section position setting unit 7222. The operation continuation section position setting unit 7222 includes a pathology gazing section setting unit 7222a that sets, as edge point images, edge points of a section in which pathologic region presence information provided preceding and following the base point image indicates present determination, and a time section setting unit 7222b that sets, as edge point images, endoscopic images at pre-decided predetermined value positions preceding and following the base point image in the section in which pathologic region presence information represents present determination.

According to the first modified of the first embodiment described above, analysis of the characteristic (feature) of a pathologic region that has been obtained as input information is performed, an extraction condition is set based on the characteristic (feature) of the pathologic region, and a high-quality image is extracted from a reference range image based on the extraction condition, whereby an endoscopic image appropriate for diagnosis can be extracted from an endoscopic image group arranged in chronological order.

### Second Modified Example of First Embodiment

Next, a second modified example of the first embodiment will be described. The second modified example of this first embodiment is different in the configurations of the pathology characteristic information calculation unit 713 and the gazing operation determination unit 714 according to the aforementioned first embodiment. Hereinafter, a pathology characteristic information calculation unit and a gazing operation determination unit according to the second modified example of this first embodiment will be described. In addition, the same configurations as those in the image processing apparatus 1 according to the aforementioned first embodiment are assigned the same sings, and the description thereof will be omitted.

FIG. 9 is a block diagram illustrating a configuration of a pathology characteristic information calculation unit according to the second modified example of the first embodiment. Based on the pathologic region information, the pathology characteristic information calculation unit 713b illustrated in FIG. 9 calculates pathology characteristic information representing a characteristic of a pathologic region. In addition, the pathology characteristic information calculation unit 713b includes a consecutive number acquisition unit 7133 that counts the number of endoscopic images starting from an endoscopic image that first includes a pathologic region, and regards the counted number as a consecutive number.

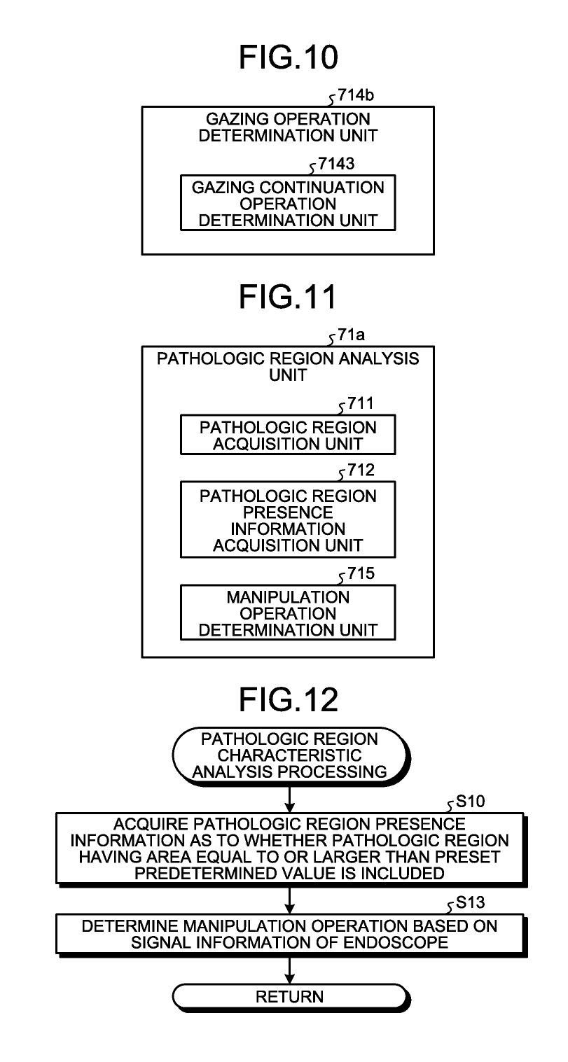

FIG. 10 is a block diagram illustrating a configuration of a gazing operation determination unit according to the second modified example of the first embodiment. A gazing operation determination unit 714b illustrated in FIG. 10 determines a gazing operation on a pathologic region based on the pathology characteristic information. In addition, the gazing operation determination unit 714b includes a gazing continuation operation determination unit 7143 that determines that gazing is to be continued, when a consecutive number in pathology characteristic information is equal to or larger than a preset predetermined value. Here, the predetermined value used for determining that gazing is to be continued, in the gazing continuation operation determination unit 7143 is a threshold for determining repetitive gazing such as every n images. In addition, among images having a consecutive number that is a predetermined number or more, images having an accumulated change amount that is equal to or less than a predetermined value are determined to be continuously gazed.

According to the second modified of the first embodiment described above, analysis of the characteristic (feature) of a pathologic region that has been obtained as input information is performed, an extraction condition is set based on the characteristic (feature) of the pathologic region, and a high-quality image is extracted from a reference range image based on the extraction condition, whereby an endoscopic image appropriate for diagnosis can be extracted from an endoscopic image group arranged in chronological order.

### Third Modified Example of First Embodiment

Next, a third modified example of the first embodiment will be described. The third modified example of the first embodiment is different in configuration of the pathologic region analysis unit 71 according to the aforementioned first embodiment and pathologic region characteristic analysis processing performed by the pathologic region analysis unit 71. Hereinafter, after a pathologic region analysis unit according to the third modified example of this first embodiment will be described, pathologic region characteristic analysis processing executed by the pathologic region analysis unit will be described. In addition, the same configurations as those in the image processing apparatus 1 according to the aforementioned first embodiment are assigned the same sings, and the description thereof will be omitted.

FIG. 11 is a block diagram illustrating a configuration of a pathologic region analysis unit according to the third modified example of the first embodiment. A pathologic region analysis unit 71a illustrated in FIG. 11 receives input of an endoscopic image group acquired by the image acquisition unit 2 via the control unit 6 or the recording unit 5, and pathologic region information representing coordinate information of a pathologic region in each endoscopic image, and analyzes the characteristic (feature) of a pathologic region in each endoscopic image. The pathologic region analysis unit 71a includes the pathologic region acquisition unit 711, the pathologic region presence information acquisition unit 712, and a manipulation operation determination unit 715 that determines a manipulation operation of an endoscope based on signal information of the endoscope.

Next, the pathologic region characteristic analysis processing executed by the pathologic region analysis unit 71a will be described. FIG. 12 is a flowchart illustrating an overview of the pathologic region characteristic analysis processing executed by the pathologic region analysis unit 71a. In FIG. 12, the pathologic region analysis unit 71a executes Step S13 in place of Steps S11 and S12 described above. Thus, the following description will be given of the steps subsequent to Step S13.

In Step S13, the manipulation operation determination unit 715 determines manipulation operation of the endoscope based on signal information of the endoscope. Specifically, the signal information of the endoscope includes image magnification ratio change information for changing a magnification ratio of an image, thumbnail acquisition information for instructing acquisition of a thumbnail (freeze image, still image), angle operation information for instructing a change of an angle, and manipulation information of other button manipulations.

According to the third modified of the first embodiment described above, analysis of the characteristic (feature) of a pathologic region that has been obtained as input information is performed, an extraction condition is set based on the characteristic (feature) of the pathologic region, and a high-quality image is extracted from a reference range image based on the extraction condition, whereby an endoscopic image appropriate for diagnosis can be extracted from an endoscopic image group arranged in chronological order.

### Fourth Modified Example of First Embodiment

Next, a fourth modified example of the first embodiment will be described. The fourth modified example of the first embodiment is different in configuration of the pathologic region analysis unit 71 and pathologic region characteristic analysis processing according to the aforementioned first embodiment. Hereinafter, after a pathologic region analysis unit according to the fourth modified example of this first embodiment will be described, pathologic region characteristic analysis processing executed by the pathologic region analysis unit will be described. In addition, the same configurations as those in the image processing apparatus 1 according to the aforementioned first embodiment are assigned the same sings, and the description thereof will be omitted.

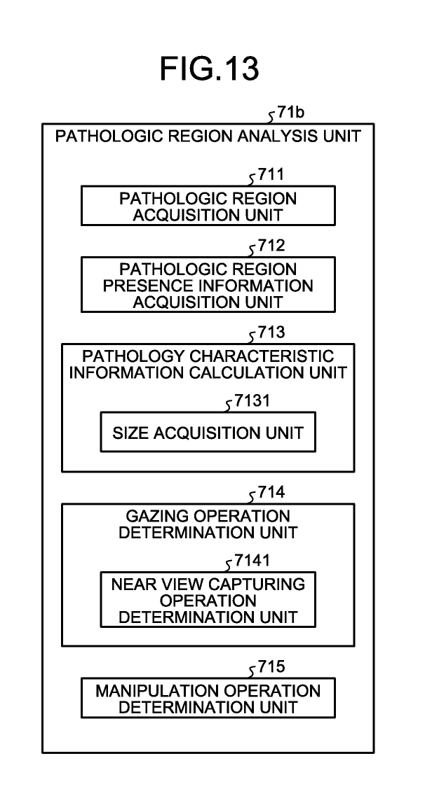

FIG. 13 is a block diagram illustrating a configuration of a pathologic region analysis unit according to the fourth modified example of the first embodiment. A pathologic region analysis unit 71b illustrated in FIG. 13 further includes the manipulation operation determination unit 715 of the pathologic region analysis unit 71a according to the third modified example of the aforementioned first embodiment, in addition to the configuration of the pathologic region analysis unit 71 according to the aforementioned first embodiment.

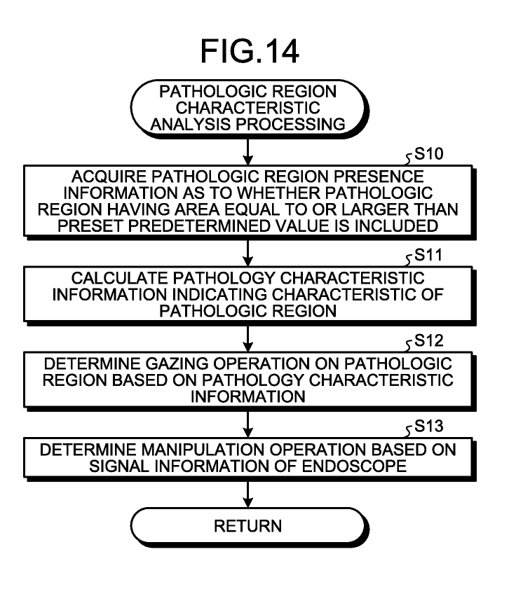

Next, the pathologic region characteristic analysis processing executed by the pathologic region analysis unit 71b will be described. FIG. 14 is a flowchart illustrating an overview of the pathologic region characteristic analysis processing executed by the pathologic region analysis unit 71b. In FIG. 14, the pathologic region analysis unit 71b executes Steps S10 to S12 in FIG. 3 described above, and executes Step S13 in FIG. 12 described above.

According to the fourth modified of the first embodiment described above, analysis of the characteristic (feature) of a pathologic region that has been obtained as input information is performed, an extraction condition is set based on the characteristic (feature) of the pathologic region, and a high-quality image is extracted from a reference range image based on the extraction condition, whereby an endoscopic image appropriate for diagnosis can be extracted from an endoscopic image group arranged in chronological order.

### Second Embodiment

Next, a second embodiment will be described. This second embodiment is different in configuration of the arithmetic unit 7 of the image processing apparatus 1 according to the aforementioned first embodiment, and different in processing to be executed. Hereinafter, after the configuration of an arithmetic unit according to this second embodiment will be described, processing executed by an image processing apparatus according to this second embodiment will be described. In addition, the same configurations as those in the image processing apparatus 1 according to the aforementioned first embodiment are assigned the same sings, and the description thereof will be omitted.

### Configuration of Arithmetic Unit

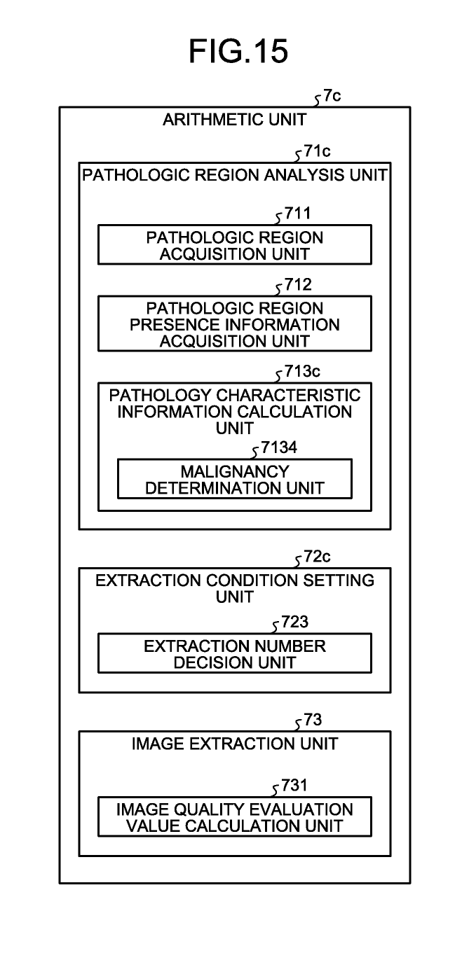

FIG. 15 is a block diagram illustrating a configuration of an arithmetic unit according to this second embodiment. An arithmetic unit 7c illustrated in FIG. 15 includes a pathologic region analysis unit 71c and an extraction condition setting unit 72c in place of the pathologic region analysis unit 71 and the extraction condition setting unit 72 according to the aforementioned first embodiment.

The pathologic region analysis unit 71c includes a pathology characteristic information calculation unit 713c in place of the pathology characteristic information calculation unit 713 according to the aforementioned first embodiment.

Based on the pathologic region information, the pathology characteristic information calculation unit 713c calculates pathology characteristic information representing a characteristic of a pathologic region. In addition, the pathology characteristic information calculation unit 713c includes a malignant degree determination unit 7134 that classifies a pathologic region according to a preset class of malignant degree.

The extraction condition setting unit 72c sets an extraction condition based on the characteristic (feature) of a pathologic region. In addition, the extraction condition setting unit 72c includes an extraction number decision unit 723 that sets, based on malignant degree information of a pathologic region, a larger number of extraction to larger malignant degree.

### Processing of Image Processing Apparatus

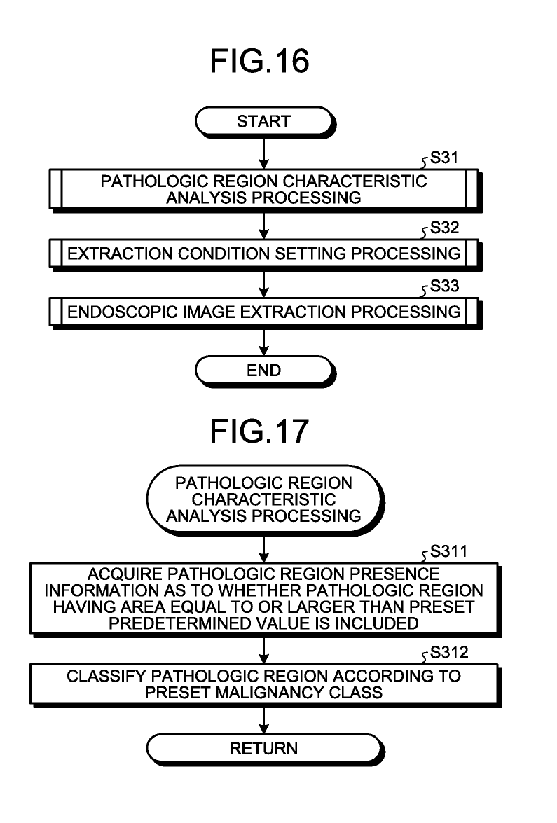

Next, an image processing method executed by the image processing apparatus 1 will be described. FIG. 16 is a flowchart illustrating an overview of processing executed by the image processing apparatus 1.

As illustrated in FIG. 16, the pathologic region analysis unit 71c acquires an endoscopic image group recorded in the recording unit 5 and pathologic region information, and executes pathologic region characteristic analysis processing of analyzing the characteristic (feature) of a pathologic region in each endoscopic image (Step S31).

FIG. 17 is a flowchart illustrating an overview of the pathologic region characteristic analysis processing in Step S31 in FIG. 16. In FIG. 17, Step S311 corresponds to Step S10 in FIG. 3 described above.

In Step S5312, the malignant degree determination unit 7134 classifies a pathologic region according to a preset class of malignant degree. Specifically, in malignant degree class classification processing, a rectangle region is set in a pathologic region, texture feature data in the rectangle region is calculated, and class classification is performed by machine learning. Here, texture feature data is calculated using a known technique such as SIFT feature data, LBP feature data, and CoHoG. Subsequently, texture feature data is vector-quantized using BoF, BoVM, or the like. In addition, in the machine learning, classification is performed using a strong classifier such as SVM. For example, pathology is classified into hyperplastic polyp, adenoma pathology, invasive cancer, and the like. After Step S312, the image processing apparatus 1 returns to a main routine in FIG. 16.

Referring back to FIG. 16, the description subsequent to Step S32 will be continued.

In Step S32, the extraction condition setting unit 72c executes, on an endoscopic image group, extraction condition setting processing of setting an extraction target range of extracting a base point and edge points decided based on the base point, based on the characteristic (feature) of the pathologic region.

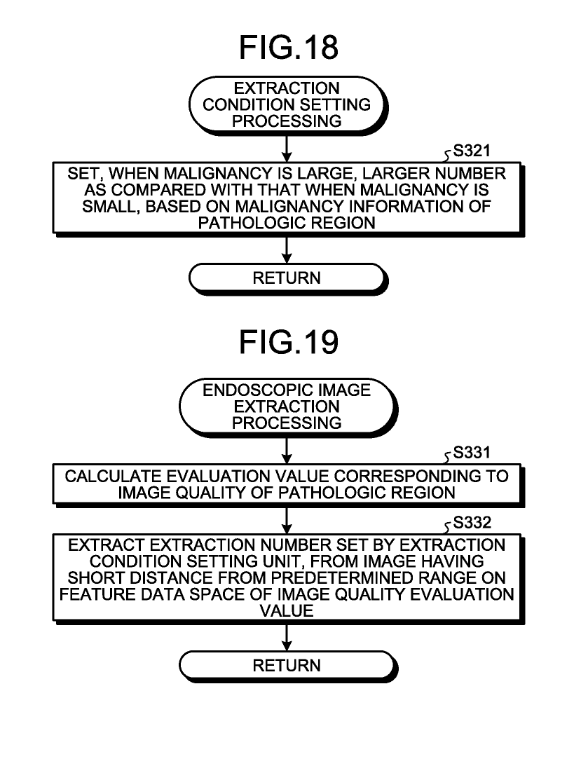

FIG. 18 is a flowchart illustrating an overview of the extraction condition setting processing in Step S32 in FIG. 16. As illustrated in FIG. 18, the extraction number decision unit 723 sets, based on malignant degree information of a pathologic region, a larger number of extraction to larger malignant degree (Step S321). After Step S321, the image processing apparatus 1 returns to a main routine in FIG. 16.

Referring back to FIG. 16, the description subsequent to Step S33 will be continued.

In Step S33, the image extraction unit 73 executes endoscopic image extraction processing of extracting, based on an extraction condition, an endoscopic image having image quality appropriate for diagnosis (image quality satisfying a predetermined condition).

FIG. 19 is a flowchart illustrating an overview of the endoscopic image extraction processing in Step S33 in FIG. 16. As illustrated in FIG. 19, the image extraction unit 73 calculates an evaluation value corresponding to the image quality of a pathologic region (Step S331). Specifically, the image extraction unit 73 acquires an evaluation value regarding image quality that is calculated similarly to Step S3 in FIG. 2 of the aforementioned first embodiment, and an evaluation value regarding malignant degree information.

Subsequently, the image extraction unit 73 extracts images by a number of extraction set by the extraction condition setting unit 72c in order from an image having a short distance from a predetermined range on a feature data space of an image quality evaluation value (Step S332). Specifically, the image extraction unit 73 extracts images by a number of extraction set by the extraction condition setting unit 72c in order from an image having a short distance from a predetermined range on a feature data space of an image quality evaluation value. After Step S332, the image processing apparatus 1 returns to a main routine in FIG. 16.

According to the second embodiment described above, analysis of the characteristic (feature) of a pathologic region that has been obtained as input information is performed, an extraction condition is set based on the characteristic (feature) of the pathologic region, and a high-quality image is extracted from a reference range image based on the extraction condition, whereby an endoscopic image appropriate for diagnosis can be extracted from an endoscopic image group arranged in chronological order.

### Third Embodiment

Next, a third embodiment will be described. This third embodiment is different in configuration of the arithmetic unit 7 of the image processing apparatus 1 according to the aforementioned first embodiment, and different in processing to be executed. Hereinafter, after the configuration of an arithmetic unit according to this third embodiment will be described, processing executed by an image processing apparatus according to this third embodiment will be described. In addition, the same configurations as those in the image processing apparatus 1 according to the aforementioned first embodiment are assigned the same sings, and the description thereof will be omitted.

### Configuration of Arithmetic Unit

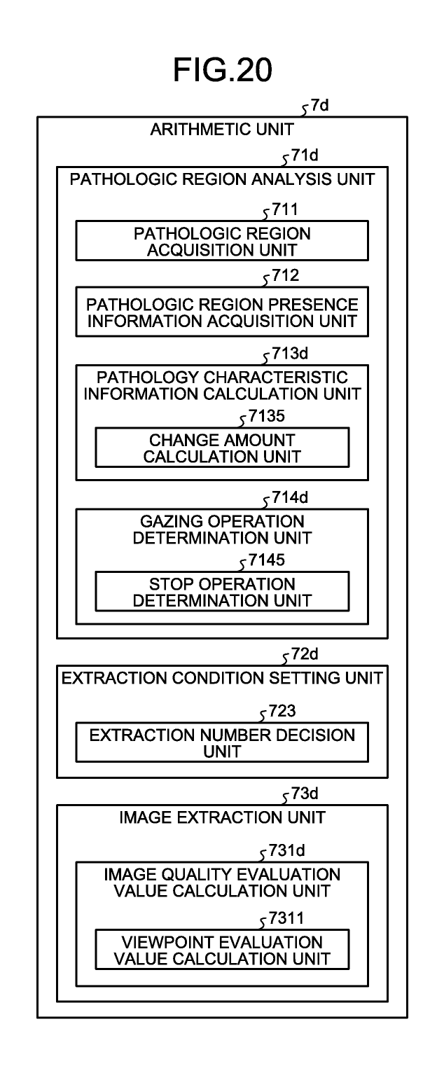

FIG. 20 is a block diagram illustrating a configuration of an arithmetic unit according to this third embodiment. An arithmetic unit 7d illustrated in FIG. 20 includes a pathologic region analysis unit 71d, an extraction condition setting unit 72d, and an image extraction unit 73d in place of the pathologic region analysis unit 71, the extraction condition setting unit 72, and the image extraction unit 73 according to the aforementioned first embodiment.

The pathologic region analysis unit 71d includes a pathology characteristic information calculation unit 713d and a gazing operation determination unit 714d in place of the pathology characteristic information calculation unit 713 and the gazing operation determination unit 714 of the pathologic region analysis unit 71 according to the aforementioned first embodiment.

Based on the pathologic region information, the pathology characteristic information calculation unit 713d calculates pathology characteristic information representing a characteristic of a pathologic region. In addition, the pathology characteristic information calculation unit 713d includes a change amount calculation unit 7135 that calculates a change amount of pathologic regions between an endoscopic image of interest and an endoscopic image adjacent to the endoscopic image of interest in chronological order, when pathologic region presence information represents present determination.

The gazing operation determination unit 714d determines a gazing operation on a pathologic region based on the pathology characteristic information. In addition, the gazing operation determination unit 714d includes a stop operation determination unit 7145 that determines to stop, when the change amount is less than a preset predetermined value.

The extraction condition setting unit 72d has the same configuration as the extraction condition setting unit 72c according to the aforementioned second embodiment, and sets an extraction condition based on the characteristic (feature) of the pathologic region. In addition, the extraction condition setting unit 72c includes an extraction number decision unit 723 that sets, based on malignant degree information of a pathologic region, a larger number of extraction to larger malignant degree.

The image extraction unit 73d extracts an endoscopic image having predetermined condition image quality, based on the extraction condition. In addition, the image extraction unit 73d includes an image quality evaluation value calculation unit 731d that calculates an evaluation value corresponding to the image quality of a pathologic region. In addition, the image quality evaluation value calculation unit 731d includes a viewpoint evaluation value calculation unit 7311 that calculates an evaluation value corresponding to viewpoint information for a pathologic region.

### Processing of Image Processing Apparatus

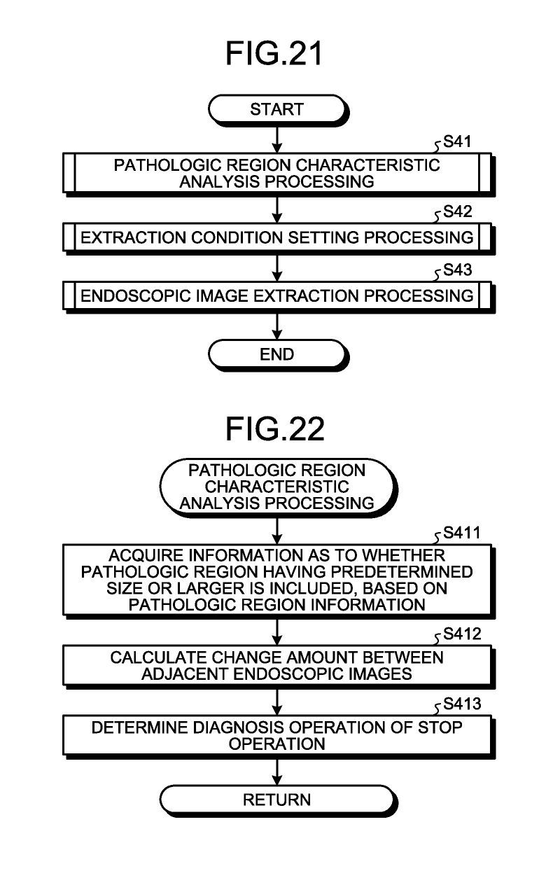

Next, an image processing method executed by the image processing apparatus 1 will be described. FIG. 21 is a flowchart illustrating an overview of processing executed by the image processing apparatus 1.

As illustrated in FIG. 21, the pathologic region analysis unit 71d acquires an endoscopic image group recorded in the recording unit 5 and pathologic region information, and executes pathologic region characteristic analysis processing of analyzing the characteristic (feature) of a pathologic region in each endoscopic image (Step S41).

FIG. 22 is a flowchart illustrating an overview of the pathologic region characteristic analysis processing in Step S41 in FIG. 21. As illustrated in FIG. 22, based on an endoscopic image group being input information acquired from the recording unit 5 by the pathologic region acquisition unit 711, and pathologic region information having coordinate information of the pathologic region, the pathologic region presence information acquisition unit 712 acquires pathologic region presence information as to whether a pathologic region having a predetermined size or larger is included, and performs determination (Step S411).

Subsequently, the change amount calculation unit 7135 calculates a change amount of pathologic regions between an endoscopic image of interest and an endoscopic image adjacent to the endoscopic image of interest in chronological order, when pathologic region presence information represents present determination (Step S412).

After that, the stop operation determination unit 7145 determines diagnosis operation of a stop operation (Step S413). Specifically, the stop operation determination unit 7145 determines to stop, when the change amount is less than a preset predetermined value. After Step S413, the image processing apparatus 1 returns to a main routine in FIG. 21.

Referring back to FIG. 21, the description subsequent to Step S42 will be continued.

In Step S42, the extraction condition setting unit 72d executes, on an endoscopic image group, extraction condition setting processing of setting an extraction target range of extracting a base point and edge points decided based on the base point, based on the characteristic (feature) of the pathologic region.

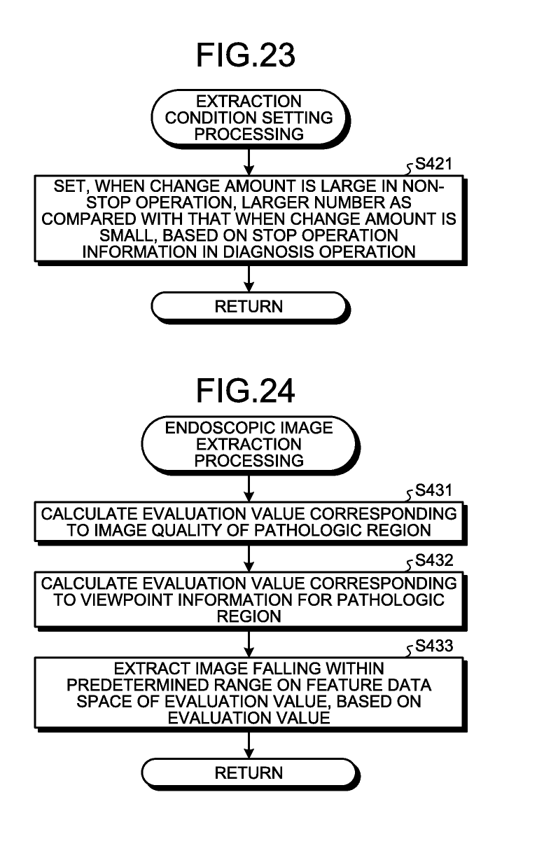

FIG. 23 is a flowchart illustrating an overview of the extraction condition setting processing in Step S42 in FIG. 21. As illustrated in FIG. 23, when a change amount is large in a non-stop operation, the extraction number decision unit 723 sets, based on stop operation information in a diagnosis operation, a larger number of extraction to a larger change amount (Step S421). After Step S421, the image processing apparatus 1 returns to a main routine in FIG. 21.

Referring back to FIG. 21, the description subsequent to Step S43 will be continued.

In Step S43, the image extraction unit 73 executes endoscopic image extraction processing of extracting, based on an extraction condition, an endoscopic image having image quality appropriate for diagnosis (image quality satisfying a predetermined condition).

FIG. 24 is a flowchart illustrating an overview of the endoscopic image extraction processing in Step S43 in FIG. 21. In FIG. 24, Steps S431 and S433 respectively correspond to Steps S331 and S332 in FIG. 19 described above. Thus, the description will be omitted.

In Step S432, the viewpoint evaluation value calculation unit 7311 calculates an evaluation value corresponding to viewpoint information for a pathologic region. Specifically, the viewpoint evaluation value calculation unit 7311 calculates an evaluation value of an image in which an important region largely appears, to be higher, such as a viewpoint viewed from the above in which a top portion of pathology can be checked, and a viewpoint viewed from the side surface in which rising of pathology can be checked. Here, the viewpoint information is defined according to inclination upside of a mucosal surface around the pathologic region. For example, the viewpoint evaluation value calculation unit 7311 calculates an evaluation value in such a manner that inclination intensity and direction of a pathology neighbor region vary, if the viewpoint is upper viewpoint. After Step S432, the image processing apparatus 1 advances the processing to Step S433.

According to the third embodiment described above, analysis of the characteristic (feature) of a pathologic region that has been obtained as input information is performed, an extraction condition is set based on the characteristic (feature) of the pathologic region, and a high-quality image is extracted from a reference range image based on the extraction condition, whereby an endoscopic image appropriate for diagnosis can be extracted from an endoscopic image group arranged in chronological order.

### Other Embodiments

In the present disclosure, an image processing program recorded in a recording device can be implemented by being executed in a computer system such as a personal computer and a work station. In addition, such a computer system may be used by being connected to a device such as another computer system or a server via a local area network (LAN), a wide area network (WAN), or a public line such as the internet. In this case, the image processing apparatus according to the first to second embodiments and the modified examples thereof may acquire image data of an intraluminal image via these networks, output an image processing result to various types of output devices such as a viewer and a printer connected via these networks, and store an image processing result into a recording device connected via these networks, such as a recording medium readable by a reading device connected to a network, for example.

In addition, in the description of the flowcharts in this specification, an anteroposterior relationship in processing between steps is clearly indicated using wordings such as “first”, “after that”, and “subsequently”, but the order of processes necessary for implementing the present disclosure is not uniquely defined by these wordings. In other words, the order of processes in the flowcharts described in this specification can be changed without causing contrariety.

According to the present disclosure, there is caused such an effect that a high-quality endoscopic image appropriate for diagnosis can be extracted from an endoscopic image group.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus comprising:
a processor comprising hardware, wherein the processor is configured to execute:
analyzing a characteristic of a pathologic region included in individual endoscopic images of an endoscopic image group arranged in chronological order;
setting, based on the characteristic of the pathologic region, an extraction condition for extracting one or more endoscopic images appropriate for diagnosis from the endoscopic image group; and
extracting, based on the extraction condition, one or more endoscopic images having image quality appropriate for diagnosis from the endoscopic image group,
wherein, when performing the analysis of the characteristic of the pathologic region, the processor
acquires pathologic region information representing coordinate information of a pathologic region in each endoscopic image of the endoscopic image group, the pathologic region information being generated by detecting a pathologic region by a pathologic region detection device from each endoscopic image of the endoscopic image group,
acquires, based on the pathologic region information, pathologic region presence information representing whether a pathologic region having an area equal to or larger than a preset predetermined value is included in each endoscopic image,
calculates, based on the pathologic region information, pathology characteristic information representing a characteristic of the pathologic region, and
classifies the pathologic region into a preset class of malignant degree.

2. The image processing apparatus according to claim 1, wherein
the processor sets, as the extraction condition, the number of extraction of the endoscopic images depending on the characteristic of the pathologic region, and
when performing the setting of the number of extraction, the processor sets a larger number of extraction to larger malignant degree.

3. An image processing method executed by an image processing apparatus, the image processing method comprising:
analyzing a characteristic of a pathologic region included in individual endoscopic images of an endoscopic image group arranged in chronological order;
setting, based on the characteristic of the pathologic region, an extraction condition for extracting one or more endoscopic images appropriate for diagnosis from the endoscopic image group; and
extracting, based on the extraction condition, an endoscopic image having image quality appropriate for diagnosis from the endoscopic image group,
wherein, in analyzing the characteristic of the pathologic region,
acquiring pathologic region information representing coordinate information of a pathologic region in each endoscopic image of the endoscopic image group, the pathologic region information being generated by detecting a pathologic region by a pathologic region detection device from each endoscopic image of the endoscopic image group,
acquiring, based on the pathologic region information, pathologic region presence information representing whether a pathologic region having an area equal to or larger than a preset predetermined value is included in each endoscopic image,
calculating, based on the pathologic region information, pathology characteristic information representing a characteristic of the pathologic region, and
classifying the pathologic region into a preset class of malignant degree.

4. A non-transitory computer-readable recording medium on which an executable program is recorded, the program instructing a processor of an image processing apparatus to execute:
analyzing a characteristic of a pathologic region included in individual endoscopic images of an endoscopic image group arranged in chronological order;
setting, based on the characteristic of the pathologic region, an extraction condition for extracting one or more endoscopic images appropriate for diagnosis from the endoscopic image group; and
extracting, based on the extraction condition, an endoscopic image having image quality appropriate for diagnosis from the endoscopic image group,
wherein, in analyzing the characteristic of the pathologic region,
acquiring pathologic region information representing coordinate information of a pathologic region in each endoscopic image of the endoscopic image group, the pathologic region information being generated by detecting a pathologic region by a pathologic region detection device from each endoscopic image of the endoscopic image group,
acquiring, based on the pathologic region information, pathologic region presence information representing whether a pathologic region having an area equal to or larger than a preset predetermined value is included in each endoscopic image,
calculating, based on the pathologic region information, pathology characteristic information representing a characteristic of the pathologic region, and
classifying the pathologic region into a preset class of malignant degree.

5. An image processing apparatus comprising:
a processor comprising hardware, wherein the processor is configured to execute:
analyzing a characteristic of a pathologic region included in individual endoscopic images of an endoscopic image group arranged in chronological order;
setting, based on the characteristic of the pathologic region, an extraction condition for extracting one or more endoscopic images appropriate for diagnosis from the endoscopic image group; and
extracting, based on the extraction condition, one or more endoscopic images having image quality appropriate for diagnosis from the endoscopic image group.

6. The image processing apparatus according to claim 5, wherein, when performing the analysis of the characteristic of the pathologic region, the processor
acquires pathologic region information representing coordinate information of a pathologic region in each endoscopic image,
acquires, based on the pathologic region information, pathologic region presence information representing whether a pathologic region having an area equal to or larger than a preset predetermined value is included in each endoscopic image,
calculates, based on the pathologic region information, pathology characteristic information representing a characteristic of the pathologic region, and
determines, based on the pathology characteristic information, a gazing operation on the pathologic region.

7. The image processing apparatus according to claim 6, wherein the processor
acquires size information of the pathologic region based on the pathologic region information when the pathologic region presence information includes information including a pathologic region having an area equal to or larger than a preset predetermined value, and
when the size information is equal to or larger than a preset predetermined value, determines that gazing is being performed with near view capturing.

8. The image processing apparatus according to claim 6,
wherein the processor
calculates a change amount in the pathologic region between an endoscopic image of interest and an endoscopic image adjacent to the endoscopic image of interest in chronological order when the pathologic region presence information includes information including a pathologic region having an area equal to or larger than a preset predetermined value, and
determines to stop when the change amount is less than a preset predetermined value.

9. The image processing apparatus according to claim 6, wherein the processor
counts the number of images starting from an image that first includes a pathologic region in each endoscopic image of the endoscopic image group, and
determines that gazing is continued when the counted number is equal to or larger than a preset predetermined value.

10. The image processing apparatus according to claim 9, wherein the predetermined value is a threshold used for determining repetitive gazing for every preset number.

11. The image processing apparatus according to claim 9, wherein the processor determines that gazing is continued on images having an accumulated change amount of the pathologic region that is less than a predetermined value among images having the counted number equal to or larger than the predetermined value.

12. The image processing apparatus according to claim 5, wherein, when performing the analysis of the characteristic of the pathologic region, the processor
acquires pathologic region information representing coordinate information of a pathologic region in each endoscopic image,
acquires, based on the pathologic region information, pathologic region presence information representing whether a pathologic region having an area equal to or larger than a preset predetermined value is included in each endoscopic image, and
determines a manipulation operation of an endoscope based on signal information of an endoscope.

13. The image processing apparatus according to claim 12, wherein the processor sets, based on the characteristic of the pathologic region, an extraction target range that is a range between a base point and each edge point decided based on the base point.

14. The image processing apparatus according to claim 13, wherein the processor sets an endoscopic image at a specific operation position as a base point image based on operation information in the characteristic of the pathologic region, and
sets, as edge point images, endoscopic images at specific operation positions preceding and following the base point image, and set a section from the base point image to the edge point images based on operation information in the characteristic of the pathologic region.

15. The image processing apparatus according to claim 14, wherein the processor sets, as the base point image, an endoscopic image in a predetermined range from a position at which a specific operation switches to another operation after the specific operation has continued for a preset predetermined section.

16. The image processing apparatus according to claim 15, wherein the processor sets, as the base point image, an endoscopic image in a predetermined range from a timing at which the operation information switches from gazing to non-gazing.

17. The image processing apparatus according to claim 15, wherein the processor sets, as the base point image, an endoscopic image in a predetermined range from a timing at which the operation information switches from near view capturing to distant view capturing.

18. The image processing apparatus according to claim 15, wherein the processor sets, as the base point image, an endoscopic image in a predetermined range from a timing at which content of the operation information switches from moving to stopping.

19. The image processing apparatus according to claim 15, wherein the processor sets, as the base point image, an endoscopic image before a timing at which a diagnosis operation switches from stopping to moving.

20. The image processing apparatus according to claim 14, wherein the processor sets, as the base point image, a point at which a specific diagnosis operation occurs.

21. The image processing apparatus according to claim 20, wherein, when the operation information is an image acquisition operation, the processor sets endoscopic images at a start time point and an end time point of manipulation of a manipulator as the base point image.

22. The image processing apparatus according to claim 14, wherein the processor sets a section up to an image in which a specific operation occurs.

23. The image processing apparatus according to claim 22, wherein the processor sets, as the edge point image, the base point images preceding and following the base point image in a section being a present section in which the pathologic region presence information includes information including a pathologic region having an area equal to or larger than a preset predetermined value.

24. The image processing apparatus according to claim 14, wherein the processor sets a section in which a specific operation is continued.

25. The image processing apparatus according to claim 24, wherein the processor sets, as the edge point image, an endoscopic image at an edge point of a section in which the pathologic region presence information preceding and following the base point image indicates present determination.

26. The image processing apparatus according to claim 24, wherein the processor sets, as the edge point image, endoscopic images at pre-decided predetermined value positions preceding and following the base point image in a section in which the pathologic region presence information includes information including a pathologic region having an area equal to or larger than a preset predetermined value.

27. The image processing apparatus according to claim 5, wherein
the processor sets, as the extraction condition, the number of extraction of the endoscopic images depending on the characteristic of the pathologic region, and
when performing the setting of the number of extraction, the processor sets a larger number of extraction to a larger change amount of the pathologic region.

28. The image processing apparatus according to claim 5, wherein the processor calculates an evaluation value corresponding to image quality of the pathologic region.

29. The image processing apparatus according to claim 28, wherein the image quality is at least any one of a color shift amount, sharpness, and an effective region area in a surface structure.

30. The image processing apparatus according to claim 28, wherein the processor calculates an evaluation value corresponding to viewpoint for the pathologic region.

31. The image processing apparatus according to claim 27, wherein the processor extracts endoscopic images by the number of extraction in order from an image having a short distance from a predetermined range on a feature data space of an image quality evaluation value.

32. The image processing apparatus according to claim 5, wherein the processor extracts an endoscopic image falling within a predetermined range on a feature data space of an image quality evaluation value.

33. The image processing apparatus according to claim 6, wherein the pathologic region information is generated by detecting a pathologic region by a pathologic region detection device from each endoscopic image of the endoscopic image group.

34. An image processing method executed by an image processing apparatus, the image processing method comprising:
analyzing a characteristic of a pathologic region included in individual endoscopic images of an endoscopic image group arranged in chronological order;
setting, based on the characteristic of the pathologic region, an extraction condition for extracting one or more endoscopic images appropriate for diagnosis from the endoscopic image group; and
extracting, based on the extraction condition, an endoscopic image having image quality appropriate for diagnosis from the endoscopic image group.

35. A non-transitory computer-readable recording medium on which an executable program is recorded, the program instructing a processor of an image processing apparatus to execute:
analyzing a characteristic of a pathologic region included in individual endoscopic images of an endoscopic image group arranged in chronological order;
setting, based on the characteristic of the pathologic region, an extraction condition for extracting one or more endoscopic images appropriate for diagnosis from the endoscopic image group; and
extracting, based on the extraction condition, an endoscopic image having image quality appropriate for diagnosis from the endoscopic image group.
